(19) United States
(12) Patent Application Publication
(10) Pub. No.: US 2008/0230072 A1
(43) Pub. Date: Sep. 25, 2008

(54) Naso-gastric mask and method

(76) Inventors: Offord L. Rollins, North Las Vegas, NV (US); Robert R. Gao, Henderson, NV (US)

Correspondence Address: WEISS & MOY PC, 4204 NORTH BROWN AVENUE, SCOTTSDALE, AZ, 85251, US

(21) Appl. No.: 11/725,455

(22) Filed: Mar. 20, 2007

(51) Int. Cl. A61M 16/06

(52) U.S. Cl. 128/207.15

(57) ABSTRACT

A mask for delivery of gas, such as oxygen, to a patient and useable in a naso-gastric intubation procedure without disruption of the seal between the mask and the face of the patient. The body of the mask includes an opening through which the naso-gastric tube may be inserted. The opening comprises a vertical lining and first and second coupling members on either side thereof and above a peripheral edge of the mask, which may be closed below the tube to provide an enhanced seal for the mask. The coupling members may comprise hook and loop members, a snap, a button, or interlocking members.

### FIELD OF THE INVENTION

The present invention relates to oxygen masks and, more particularly, to an improved naso-gastric oxygen mask providing an improved sealing relationship between the naso-gastric tube and mask.

### BACKGROUND OF THE INVENTION

A wide variety of oxygen masks have been available, varying in construction, style, and material, depending upon the specific purpose for which each is desired to be employed. The basic mask which is available today uses neither a valve nor a reservoir bag. Exhaled air from the lungs of the patient is usually vented through holes in the body of the mask. In view of its convenience and relative comfort, the basic mask is widely used whenever moderate oxygen concentrations are desired for short periods of time. This might occur, for example, during the postoperative recovery state of a patient. Such a mask might also be used, for example, during either temporary or interim therapy when a patient is being weaned from continuous oxygen administration.

In many postoperative and related cases, a naso-gastric intubation procedure is necessary. In such a case, plastic tubing is usually inserted into the patient's nasal passageway and guided down the esophagus into the upper gastric area. This tubing is an obstruction, as far as the administration of oxygen is concerned, and complicates the application of the mask.

In the past, if a naso-gastric tube and an oxygen mask were to be used simultaneously, the tube was put in place first and the mask is then applied. The seal of the mask against the face of the patient was incomplete due the protrusion of the tube at the point that the tube intersected the body of the mask. In other words, it was impossible to conform the mask to the facial configuration of the patient and, in many cases, the mask was generally askew. Such incorrect seating of the mask allowed oxygen to freely pass to the atmosphere, resulting in treatment of the patient with a decreased and uncontrolled volume.

Additionally, the stability of the mask as well as the patient's comfort were complicated by the tube. The mask was much less secure and more easily dislodged by an unobservant, restless, or mobile patient. Also, the tube was usually placed across and secured to the facial skin in an attempt to prevent relative movement among the patient, tube, and mask. The taping of the tube to the skin often produced discomfort and runs the risk of producing a pressure necrosis of the skin.

U.S. Pat. No. 4,328,797, issued to one of the applicants herein, provided one solution to these problems. More specifically, it was directed to an oxygen-administration device which could be simultaneously employed with a naso-gastric tube in such a manner that the volume of oxygen can be controlled at least to the same extent as may be attained with a fully seated oxygen mask. While U.S. Pat. No. 4,328,797 addressed the problem of permitting a naso-gastric tube to be inserted through an oxygen mask without disturbing the seal of the mask to the face of the patient, the problem of discomfort caused by contact between the tube and the face remained, since the tube was inserted proximate a perimeter of the mask.

### SUMMARY OF THE INVENTION

In accordance with one embodiment of the present invention, a naso-gastric mask for the administration of a gas such as oxygen is disclosed. The mask comprises, in combination: a mask body of such size and shape adapted to cover at least the nose of a patient and having a peripheral edge; a gas tube coupled to the mask; a naso-gastric tube opening on the mask body; wherein the naso-gastric tube opening has a vertical lining and a naso-gastric tube opening at an upper terminus of the vertical lining, and wherein the naso-gastric tube opening is adapted to hold a naso-gastric tube therein; a first closing member located on a first side of the vertical lining; and a second closing member located on a second side of the vertical lining.

In accordance with another embodiment of the present invention, a naso-gastric mask for the administration of a gas such as oxygen is disclosed. The mask comprises, in combination: a mask body of such size and shape adapted to cover at least the nose of a patient and having a peripheral edge; a gas tube coupled to the mask; a naso-gastric tube opening on the mask body; wherein the naso-gastric tube opening has a vertical lining and a naso-gastric tube opening at an upper terminus of the vertical lining, and wherein the naso-gastric tube opening is adapted to hold a naso-gastric tube therein; a tab located on a first side of the vertical lining and having an adhesive material thereunder and adapted to extend to seal to a second side of the vertical lining.

In accordance with a further embodiment of the present invention, a method for positioning a naso-gastric tube is disclosed. The method comprises: providing a mask body of such size and shape adapted to cover at least the nose of a patient and having a peripheral edge; providing a gas tube coupled to the mask; providing a naso-gastric tube opening on the mask body; wherein the naso-gastric tube opening has a vertical lining and a naso-gastric tube opening at an upper terminus of the vertical lining, and wherein the naso-gastric tube opening is adapted to hold a naso-gastric tube therein; wherein the mask body has a first closing member located on a first side of the vertical lining and a second closing member located on a second side of the vertical lining; inserting a naso-gastric tube through the vertical lining and into the naso-gastric tube opening; and coupling the first closing member to the second closing member.

### BRIEF DESCRIPTION OF THE DRAWINGS

FIG. 1 is a perspective view of an oxygen mask consistent with an embodiment of the present invention, with the naso-gastric tube entry portion in the closed position.

FIG. 2 is a perspective view of an oxygen mask consistent with another embodiment of the present invention, with the naso-gastric tube entry portion in the closed position.

FIG. 3 is a perspective view of an oxygen mask consistent with a further embodiment of the present invention, with the naso-gastric tube entry portion in the closed position.

FIG. 4 is a perspective view of an oxygen mask consistent with a still further embodiment of the present invention, with the naso-gastric tube entry portion in the closed position.

FIG. 5 is a perspective view of the oxygen mask of FIG. 2, with the naso-gastric tube entry in the open position.

FIG. 6 is a perspective view of the oxygen mask of FIG. 4, with the naso-gastric tube entry in the open position.

FIG. 7 is a perspective view of an oxygen mask consistent with a still further embodiment of the present invention, with the naso-gastric tube entry portion in the open position.

FIG. 8 is a perspective view of the oxygen mask of FIG. 7, with the naso-gastric tube entry in the closed position.

FIG. 9 is a perspective view of the oxygen mask of FIG. 5, with the naso-gastric tube entry in the closed position.

FIG. 10 is a perspective view of a prior art oxygen mask.

FIG. 11 is a perspective an oxygen mask consistent with a still further embodiment of the present invention, with a plurality of naso-gastric tube entries.

### DETAILED DESCRIPTION OF THE PREFERRED EMBODIMENTS

Referring first to FIG. 10, a prior art naso-gastric oxygen mask 100 is shown. The components of the mask 100 comprise a mask body 102, strap 104, gas hose 106, and naso-gastric tube 108. It can be seen that naso-gastric tube 108 enters the mask body 102 via naso-gastric tube entry 110, which is located proximate a peripheral edge 112 of the mask body 102, near the face of a patient 114.

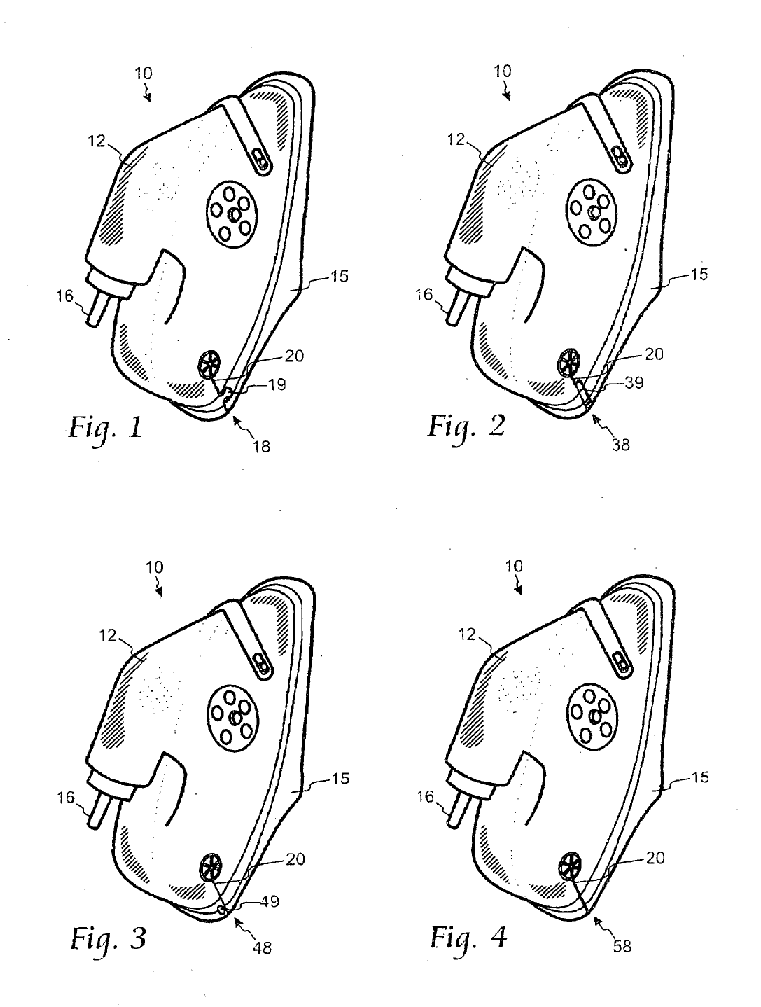

Referring now to FIG. 1, an improved naso-gastric oxygen mask 10 (herein “mask 10”) consistent with an embodiment of the present invention is shown. Like the prior art mask 100, the mask 10 includes a mask body 12 that is adapted to be positioned over a patient's mouth and nose. It further includes a gas hose 16, which transports oxygen or other gas to mask body 12 for dispersal into the interior of the mask body 12 when positioned on the face of a patient.

Located on the mask body 12 is a closeable naso-gastric tube entry 18 (“entry 18”), which permits the insertion therethrough of a naso-gastric tube (see, e.g., FIG. 8). The entry 18 comprises a vertical lining 20 sealable by adhesion of a tab 19, positioned on a first side of the vertical lining 20 and having adhesive material on an underside thereof, to a second side of the vertical lining 20. The entry 18 further comprises, at an upper terminus of the vertical lining 20, a naso-gastric tube opening adapted to hold a naso-gastric tube therein, as shown by way of example in FIG. 8. (The term “vertical” as used herein with respect to the term “lining” means that the vertical lining 20 has a substantially vertical orientation relative to the peripheral edge 15 of the mask body 12. Also, it should be noted that while the naso-gastric opening is shown, by way of example, as circumferential, other shapes may be utilized, provided that they are capable of receiving therein a naso-gastric tube.) In use, the vertical lining 20 is opened, a naso-gastric tube is inserted therethrough and into the naso-gastric tube opening, and the tab 19, with the adhesive material exposed, is then positioned on the second side of the vertical lining 20, substantially and securely sealing the vertical lining 20 with the naso-gastric tube in a sealed relationship inside the naso-gastric tube opening portion of the entry 18.

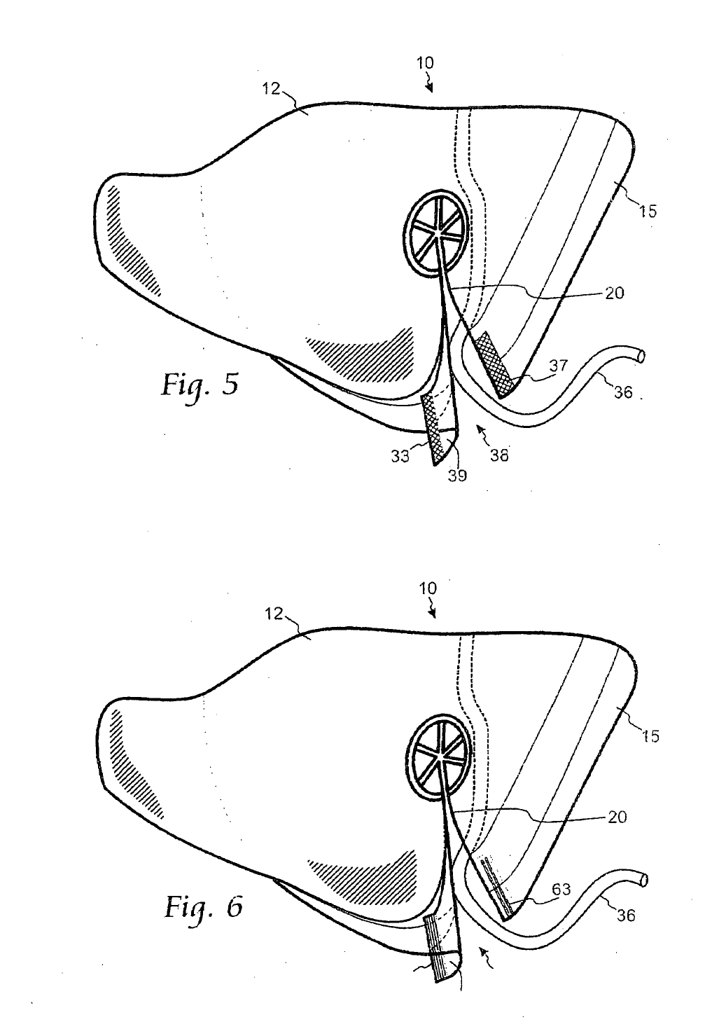

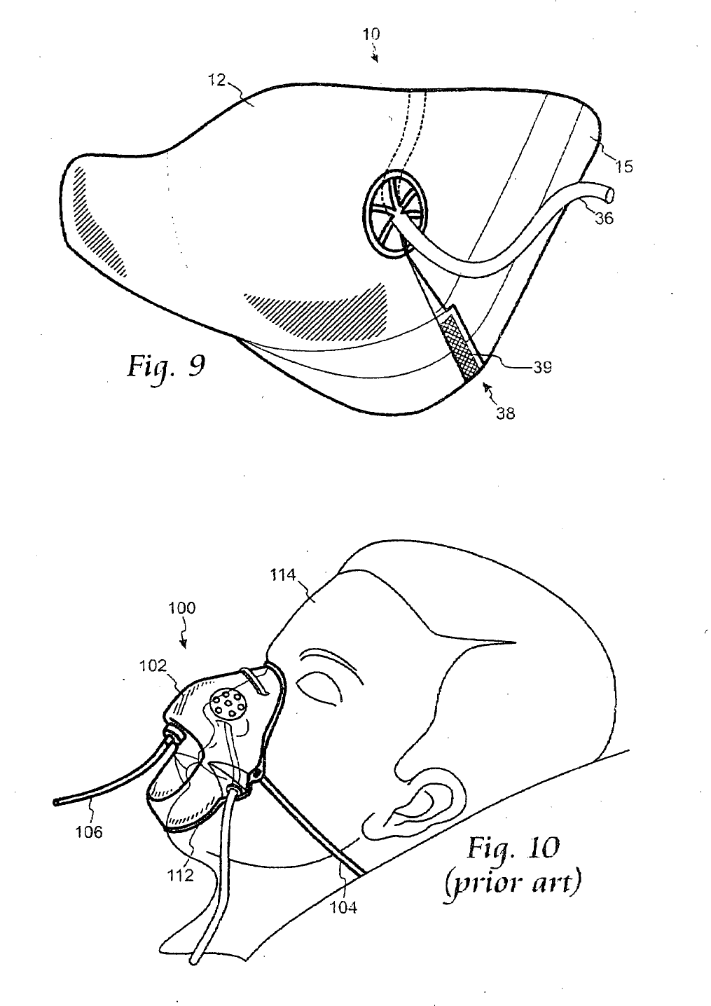

Referring now to FIGS. 2, 5 and 9, another embodiment of an improved mask 10 is illustrated. As described above with respect to the embodiment of FIG. 1, the mask 10 includes a mask body 12 that is adapted to be positioned over a patient's mouth and nose. It further includes a gas hose 16, which transports oxygen or other gas to mask body 12 for dispersal into the interior of the mask body 12 when positioned on the face of a patient.

Located on the mask body 12 is another embodiment of a closeable naso-gastric tube entry 38 (“entry 38”), which permits the insertion therethrough of a naso-gastric tube 36. The entry 38 comprises a vertical lining 20 sealable by placement of a tab 39, located on a first side of the vertical lining 20 and having one of hook and loop material 33 on an underside thereof, onto a corresponding one of hook and loop material 37 on a second side of the vertical lining 20. The entry 38 further comprises, at an upper terminus of the vertical lining 20, a naso-gastric tube opening adapted to hold a naso-gastric tube therein, as shown by way of example in FIG. 8. In use, the vertical lining 20 is opened, a naso-gastric tube is inserted therethrough and into the naso-gastric tube opening, and the material 33 on the underside of the tab 39 is then placed in contact with the material 37, below the naso-gastric tube, substantially and securely sealing the vertical lining 20 with the naso-gastric tube in a sealed relationship inside the naso-gastric tube opening portion of the entry 38.

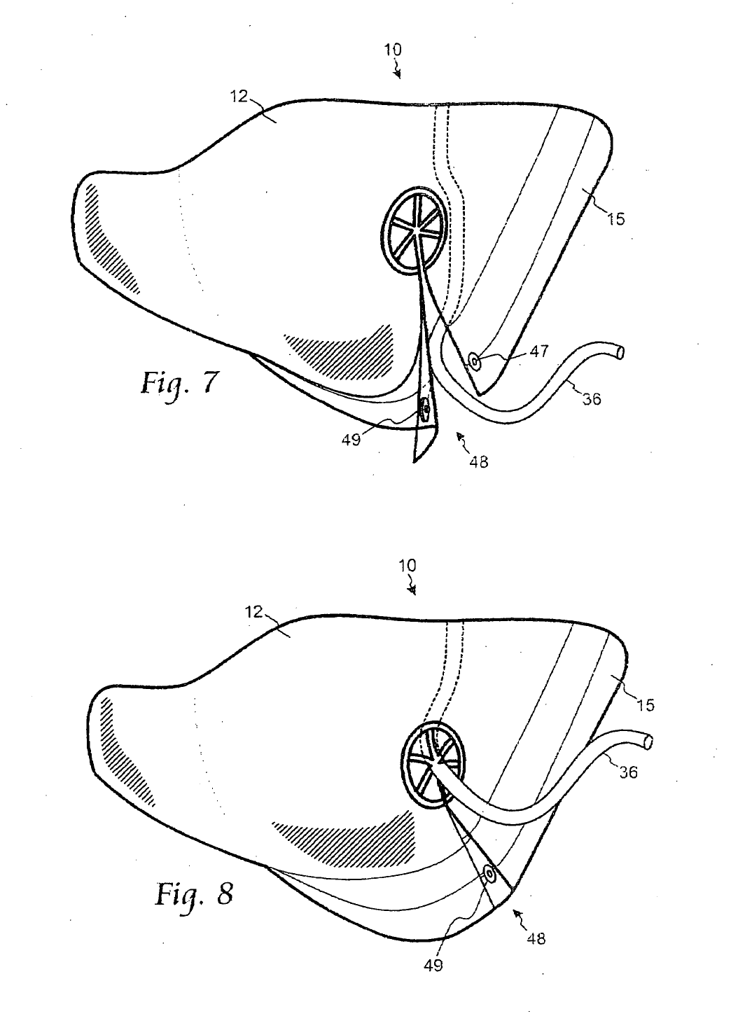

Referring now to FIGS. 3 and 7-8, another embodiment of an improved mask 10 is illustrated. As described above with respect to the embodiment of FIG. 1, the mask 10 includes a mask body 12 that is adapted to be positioned over a patient's mouth and nose. It further includes a gas hose 16, which transports oxygen or other gas to mask body 12 for dispersal into the interior of the mask body 12 when positioned on the face of a patient.

Located on the mask body 12 is another embodiment of a closeable naso-gastric tube entry 48 (“entry 48”), which permits the insertion therethrough of a naso-gastric tube 36. The entry 48 comprises a vertical lining 20 sealable by insertion of a male snap component 49 located on a first side of the vertical lining 20 into a corresponding female snap component 47 on a second side of the vertical lining 20. The entry 48 further comprises, at an upper terminus of the vertical lining 20, a naso-gastric tube opening adapted to hold a naso-gastric tube therein, as shown by way of example in FIG. 8. In use, the entry 48 is opened, a naso-gastric tube is inserted therethrough and into the naso-gastric tube opening, and the snap components 49 and 47 are closed below the naso-gastric tube, substantially and securely sealing the vertical lining 20 with the naso-gastric tube in a sealed relationship inside the naso-gastric tube opening portion of the entry 48.

Referring now to FIGS. 4 and 6, still another embodiment of an improved mask 10 is illustrated. As described above with respect to the embodiment of FIG. 1, the mask 10 includes a mask body 12 that is adapted to be positioned over a patient's mouth and nose. It further includes a gas hose 16, which transports oxygen or other gas to mask body 12 for dispersal into the interior of the mask body 12 when positioned on the face of a patient.

Located on the mask body 12 is another embodiment of a closeable naso-gastric tube entry 58 (“entry 58”), which permits the insertion therethrough of a naso-gastric tube 36. The entry 58 comprises a vertical lining 20 sealable by placement of a tab 59, located on a first side of the vertical lining 20 and having a first interlocking sealing member 61 on an underside thereof, into a corresponding second interlocking sealing member 63 on a second side of the vertical lining 20. The entry 58 further comprises, at an upper terminus of the vertical lining 20, a naso-gastric tube opening adapted to hold a naso-gastric tube therein, as shown by way of example in FIG. 8. In use, the entry 58 is opened, a naso-gastric tube 36 is inserted therethrough and into the naso-gastric tube opening, and the first and second interlocking sealing members 61 and 63 are sealed together, below the naso-gastric tube, substantially and securely sealing the vertical lining 20 with the naso-gastric tube in a sealed relationship inside the naso-gastric tube opening portion of the entry 58.

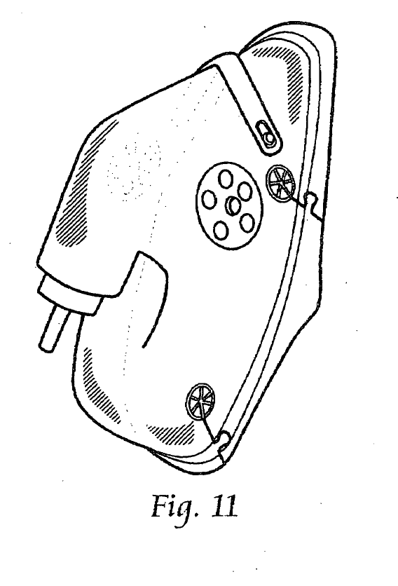

Referring now to FIG. 11, it can be seen that it may be desired to provide more than one naso-gastric tube entry, in order to provide a health care provider with increased flexibility for purposes of selecting a desired location for insertion into the mask of a naso-gastric tube.

It should be noted with respect to all of the above-described embodiments that it may be desired to utilize the mask 10 without a naso-gastric tube. For such use, it will be desired to close the naso-gastric tube entry, using the closing/sealing structure provided, to provide an enhanced seal of the entry and to reduce leakage from the interior of the mask 10.

It can be seen that an object of the present invention is to elevate the point of entry of the naso-gastric tube into the mask, above the peripheral edge of the mask. In so doing, the extent of contact between an outer surface of the naso-gastric tube and the skin of the patient, in the area inside the mask, is reduced. Such a configuration can reduce patient discomfort.

Although particular embodiments of the invention have been described in detail for purposes of illustration, various modifications may be made without departing from the spirit and scope of the invention. Accordingly, the invention is not to be limited, except as by the appended claims.

We claim:

1. A naso-gastric mask for the administration of a gas such as oxygen comprising, in combination:
a mask body of such size and shape adapted to cover at least the nose of a patient and having a peripheral edge;
a gas tube coupled to the mask;
a naso-gastric tube opening on the mask body;
wherein the naso-gastric tube opening has a vertical lining and a naso-gastric tube opening at an upper terminus of the vertical lining, and wherein the naso-gastric tube opening is adapted to hold a naso-gastric tube therein;
a first closing member located on a first side of the vertical lining; and
a second closing member located on a second side of the vertical lining.

2. The mask of claim 1 wherein the first closing member comprises a male tab member.

3. The mask of claim 2 wherein the second closing member comprises a corresponding female tab member.

4. The mask of claim 1 wherein the first closing member comprises a tab having one of hook and loop material on an underside thereof.

5. The mask of claim 4 wherein the second closing member comprises a the other of one of hook and loop material on an exposed surface thereof.

6. The mask of claim 1 wherein the first closing member comprises a male snap component.

7. The mask of claim 6 wherein the second closing member comprises a corresponding female snap component.

8. The mask of claim 1 wherein the first closing member comprises a first interlocking sealing member.

9. The mask of claim 8 wherein the second closing member comprises a corresponding second interlocking sealing member.

10. The mask of claim 1 further comprising a naso-gastric tube insertable in the circumferential naso-gastric tube opening.

11. A naso-gastric mask for the administration of a gas such as oxygen comprising, in combination:
a mask body of such size and shape adapted to cover at least the nose of a patient and having a peripheral edge;
a gas tube coupled to the mask;
a naso-gastric tube opening on the mask body;
wherein the naso-gastric tube opening has a vertical lining and a naso-gastric tube opening at an upper terminus of the vertical lining, and wherein the naso-gastric tube opening is adapted to hold a naso-gastric tube therein;
a tab located on a first side of the vertical lining and having an adhesive material thereunder and adapted to extend to seal to a second side of the vertical lining.

12. The mask of claim 11 further comprising a naso-gastric tube insertable in the circumferential opening.

13. A method for positioning a naso-gastric tube comprising:
providing a mask body of such size and shape adapted to cover at least the nose of a patient and having a peripheral edge;
providing a gas tube coupled to the mask;
providing a naso-gastric tube opening on the mask body,
wherein the naso-gastric tube opening has a vertical lining and a naso-gastric tube opening at an upper terminus of the vertical lining, and wherein the naso-gastric tube opening is adapted to hold a naso-gastric tube therein;
wherein the mask body has a first closing member located on a first side of the vertical lining and a second closing member located on a second side of the vertical lining;
inserting a naso-gastric tube through the vertical lining and into the naso-gastric tube opening; and
coupling the first closing member to the second closing member.

14. The mask of claim 13 wherein the first closing member comprises a male tab member.

15. The mask of claim 14 wherein the second closing member comprises a corresponding female tab member.

16. The mask of claim 13 wherein the first closing member comprises a tab having one of hook and loop material on an underside thereof.

17. The mask of claim 16 wherein the second closing member comprises the other of one of hook and loop material on an exposed surface thereof.

18. The mask of claim 13 wherein the first closing member comprises a male snap component.

19. The mask of claim 18 wherein the second closing member comprises a corresponding female snap component.

20. The mask of claim 13 wherein the first closing member comprises a first interlocking sealing member and the second closing member comprises a corresponding second interlocking sealing member.
